(19) United States
(12) Patent Application Publication
(10) Pub. No.: US 2025/0046451 A1
(43) Pub. Date: Feb. 6, 2025

(54) Generative Adversarial Network for Urine Biomarkers

(71) Applicant: Kidneymetrix, Inc., Brisbane, CA (US)

(72) Inventors: Wanzin YAZAR, Daly City, CA (US); Reuben SARWAL, Portola Valley, CA (US); Srinka GHOSH, San Francisco, CA (US)

(21) Appl. No.: 18/714,787

(22) PCT Filed: Nov. 23, 2022
(86) PCT No.: PCT/US2022/050974

§ 371 Date: May 30, 2024

Related U.S. Application Data
(60) Provisional application No. 63/284,590, filed on Nov. 30, 2021.

(51) Int. Cl. G16H 50/20; G16B 20/00; G16B 40/20; G16H 10/60

(52) U.S. Cl. CPC G16H 50/20; G16B 20/00; G16B 40/20; G16H 10/60

(57) ABSTRACT

Disclosed here are Generative Adversarial Network (GANs) based data augmentation methods for providing synthetic biological samples, such as urine or blood samples, in scenarios with a small imbalanced biomedical dataset for machine learning systems. In specific aspects, the disclosure provides synthetic data generated from a learned distribution of urinary analyte concentrations from real samples with corresponding biomarker data, particularly cfDNA.

### CROSS-REFERENCE

The present application claims priority to U.S. Provisional Application Ser. No. 63/284,590 filed Nov. 30, 2021, the contents of which are hereby incorporated by reference in their entirety.

### FIELD OF THE INVENTION

The present invention relates generally to methodologies for balancing imbalanced biological data set.

### BACKGROUND

Several cutting-edge artificial intelligence applications have a challenging and long-standing problem dealing with small imbalanced datasets in their implementations. The problem of class imbalance arises when there is an uneven number of samples for all classes present in a dataset, and it can cause machine learning algorithms to produce a poor performance on the minority classes while favoring bias towards the majority class. This is a common problem that affects many real-world applications such as credit card fraud detections, spam detection, churn prediction, medical diagnosis, dense object detection, amongst others. There is a pressing need for technologies that can address bias introduced in machine learning systems trained with small imbalanced datasets.

### SUMMARY

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Disclosed herein are uses and systems of Generative Adversarial Network (GANs)-based data augmentation methods to create synthetic features, particularly in scenarios with small imbalanced biomedical dataset for machine learning systems. Such a complex multivariate analysis of biomarkers from a urine sample.

In some aspects, the disclosure provides a system configured to balance an imbalanced dataset obtained from a biological sample, comprising: one or more computer subsystems; and one or more components executed by the one or more computer subsystems, wherein the one or more components comprise a generative adversarial network trained with: a first training set comprising data corresponding to an amount of cell-free DNA (cfDNA) biomarker from a subject with an organ injury designated as a first training input; a second training set comprising data corresponding to an amount of cell-free DNA (cfDNA) biomarker from a subject without the organ injury designated as a second training input; wherein the first and the second datasets are imbalanced and the one or more computer subsystems are configured for generating a set of synthetic features for the first dataset and/or the second dataset by inputting a portion of the data from the first training input and the second training input into the generative adversarial network.

In some cases, the generative adversarial network is configured as a conditional generative adversarial network, as a vanilla generative adversarial network, as a table generative adversarial network, as a tabular generative adversarial network. In some instances the generative adversarial network is further trained with an additional training set comprising data corresponding to an amount of a methylated cfDNA biomarker (m-cfDNA) from a subject with organ injury designated as an additional training input; an additional training set comprising data corresponding to an amount of a methylated cfDNA biomarker (m-cfDNA) from a subject without organ injury designated as an additional training input.

In some instances, the generative adversarial network is further trained with an additional training set comprising data corresponding to an amount of an inflammatory biomarker from a subject with organ injury designated as an additional training input; an additional training set comprising data corresponding to an amount of an inflammatory biomarker from a subject without organ injury designated as an additional training input. The inflammatory biomarker can be a member of the chemokine (C—X—C motif) ligand family, such as C—X—C motif chemokine ligand 1 (CXCL1), C—X—C motif chemokine ligand 2 (CXCL2), C—X—C motif chemokine ligand 5 (CXCL5), C—X—C motif chemokine ligand 9 (CXCL9) (MIG), or C—X—C motif chemokine ligand 10 (CXCL10) (IP-10).

In some instances, the generative adversarial network is further trained with an additional training set comprising data corresponding to an amount of an apoptosis biomarker from a subject with organ injury designated as an additional training input; an additional training set comprising data corresponding to an amount of an apoptosis biomarker from a subject without organ injury designated as an additional training input. In some instances, the apoptosis biomarker is clusterin.

In some cases, the generative adversarial network is further trained with an additional training set comprising data corresponding to an amount of a protein from a subject with organ injury designated as an additional training input; an additional training set comprising data corresponding to an amount of a protein from a subject without organ injury designated as an additional training input. In some cases, the protein is albumin, but the protein can also be total protein.

In some aspects, the one or more computer subsystems are further configured for determining one or more characteristics of the synthetic features for the first dataset and/or the second dataset. In other aspects, the one or more computer subsystems are further configured to train a machine learning model using the simulated image. Such machine learning models can be trained on the first data input, on the second data input, or on any number of data inputs. In some cases, the machine learning model is trained on the first data input and on the second data input, but not on the set of synthetic features. In some instances, the machine learning model is CTGAN, SMOTE, SVM-SMOTE, ADASYN.

In some instances the biological sample is urine, but it can also be blood, a bronchiolar lavage, or another suitable bodily fluid. In some instances, the organ is an allograft, and the injury is cause by rejection of the allograft by the subject. In some instances the organ is a kidney, a pancreas, a heart, a lung, or a liver. In some instances, the organ is a kidney. In some instances, the injury is chronic kidney injury (CKI) or acute kidney injury (AKI). In some instances, the injury is caused by a viral infection suffered by the subject such as a viral infection is caused by Sars-COV-2, CMV, or BKV. In some instances, the injury is a cancer harming the organ, such as a bladder cancer or kidney cancer. In some instances, the subject is a human.

In some aspects the disclosure provides a system configured to analyze a dataset obtained from a biological sample, comprising: one or more computer subsystems; and one or more components executed by the one or more computer subsystems, wherein the one or more components comprise a generative adversarial network trained with a training set corresponding to an amount of cfDNA from a subject; and wherein the one or more computer subsystems are configured for generating a synthetic dataset from the biological sample by inputting a subset of the training data into the generative adversarial network. In some instances, at least one subset of the training data is annotated with a biological condition, such as a biological condition of acute rejection, a biological condition of chronic kidney injury (CKI), acute kidney injury (AKI), biological condition of COVID-19, or a biological condition of healthy or stable. In some instances, the cfDNA is from a urine sample. In others the cfDNA is from a blood or plasma sample, but a variety of bodily fluids are suitable, such as saliva, bronchiolar lavage, etc.

In some instances, the generative adversarial network is further trained with an additional training set comprising data corresponding to an amount of a methylated cfDNA biomarker (m-cfDNA) from a subject, further trained with an additional training set comprising data corresponding to an amount of an inflammatory biomarker from a subject, such as a member of the chemokine (C—X—C motif) ligand family, for examples: C—X—C motif chemokine ligand 1 (CXCL1), C—X—C motif chemokine ligand 2 (CXCL2), C—X—C motif chemokine ligand 5 (CXCL5), C—X—C motif chemokine ligand 9 (CXCL9) (MIG), or C—X—C motif chemokine ligand 10 (CXCL10) (IP-10). In some instances, the generative adversarial network is further trained with an additional training set comprising data corresponding to an amount of an apoptosis biomarker from a subject, such as clusterin.

In some instances, the generative adversarial network is further trained with an additional training set comprising data corresponding to an amount of a protein, such as albumin or total protein. In some instances, the subject is a human.

In some aspects, the disclosure provides a non-transitory computer-readable medium, storing program instructions executable on one or more computer systems for performing a computer-implemented method for generating a simulated image of a specimen, wherein the computer-implemented method comprises: one or more computer subsystems; and one or more components executed by the one or more computer subsystems, wherein the one or more components comprise a generative adversarial network trained with a training set corresponding to an amount of cfDNA from a subject; and wherein the one or more computer subsystems are configured for generating a synthetic dataset from the biological sample by inputting a sub-set of the training data into the generative adversarial network.

In some aspects the disclosure provides a non-transitory computer-readable medium, storing program instructions executable on one or more computer systems for performing a computer-implemented method for generating a simulated image of a specimen, wherein the computer-implemented method comprises: one or more computer subsystems; and one or more components executed by the one or more computer subsystems, wherein the one or more components comprise a generative adversarial network trained with a training set comprising data corresponding to an amount of cell-free DNA (cfDNA) biomarker from a subject a first training set comprising data corresponding to an amount of cell-free DNA (cfDNA) biomarker from a subject with an organ injury designated as a first training input; a second training set comprising data corresponding to an amount of cell-free DNA (cfDNA) biomarker from a subject without the organ injury designated as a second training input; wherein the first and the second datasets are imbalanced and the one or more computer subsystems are configured for generating a set of synthetic features for the first dataset and/or the second dataset by inputting a portion of the data from the first training input and the second training input into the generative adversarial network.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 (FIG. 1) illustrates a traditional oversampling method (SMOTE).

FIG. 2 (FIG. 2) illustrates a strategy for enlarging training dataset with different data augmentation methods.

FIG. 3 (FIG. 3) illustrates a strategy for training different Generative Adversarial Networks (GANs); incorporating extraneous data (i.e., synthetic samples or synthetic features or extraneous data) therein, and subsequently training different algorithms.

FIG. 4A-FIG. 4H (FIGS. 4A-4H) collective illustrate a comparison between a range of time points and exemplary biomarkers measured with original biological samples (i.e., features on original biological samples) and synthetic samples (i.e., synthetic features) based on their distribution produced by CTGAN (conditional tabular generative adversarial networks).

FIG. 5A-FIG. 5H (FIGS. 5A-5H) collectively illustrate a comparison between a range of time points and exemplary biomarkers measured with original biological samples (i.e., features on original biological samples) and synthetic samples (i.e., synthetic features) based on the first two principal components produced by CTGAN.

FIG. 6A-FIG. 6B (FIGS. 6A-6B) collectively illustrate the result analysis of machine learning algorithms' performance on training samples+synthetic samples augmented by different oversampling techniques.

FIG. 7 (FIG. 7) is a tabulation of the results of the Random Forest Algorithm, XGBoost algorithm, and LightGBM algorithm trained on original data, trained on SMOTE's generated samples, trained on ADASYN's generated samples, trained on SVMSMOTE's generated samples, trained on CTGAN's generated samples. This figure demonstrates the feasibility of using a variety of strategies for augmenting samples with synthetic manner in a manner that generally reproduces the ROC-AUC obtained with the original data.

FIG. 8A-FIG. 8C (FIGS. 8A-8C) collectively illustrate illustrates the performance of a random forest model oversampled by CTGAN and a baseline (FIG. 8A), a random forest model oversampled by SVM_SMOTE and SMOTE (FIG. 8B), and a random forest model oversampled by ADASYN (FIG. 8C), on kidney transplant rejection datasets with synthetic urine samples.

FIG. 9 (FIG. 9) illustrates non-parametric results of random forest-based rejection scores using a SMOTE synthetic data generation method for providing a Q-Score. The axis of FIG. 9 represent the SMOTE generated Q-Score (Y-axis) over the SMOTE phenotype (X-axis).

FIG. 10 (FIG. 10) illustrates non-parametric results of random forest-based rejection scores using original (i.e., biological) data generation method for providing a Q-Score. The axis of FIG. 10 represent the Q-Score of the original data (Y-axis) over the original phenotype.

FIG. 11 (FIG. 11) illustrates non-parametric results of random forest-based rejection scores using a GAN synthetic data generation method for providing a Q-Score. The axis of FIG. 11 represent the GAN generated Q-Score (Y-axis) over the GAN phenotype (X-axis).

FIG. 12 (FIG. 12) illustrates non-parametric results of random forest-based rejection scores using a ADASYN synthetic data generation method for providing a Q-Score. The axis of FIG. 12 represent the ADASYN generated Q-Score (Y-axis) over the ADASYN phenotype (X-axis).

FIG. 13 (FIG. 13) illustrates non-parametric results of random forest-based rejection scores using a SVM synthetic data generation method for providing a Q-Score. The axis of FIG. 13 represent the SVM generated Q-Score (Y-axis) over the phenotype (X-axis).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTIONS

In a medical diagnosis application, information about healthy patients is much richer than those about affected ones; hence, machine learning algorithms are prone to misclassifying some unhealthy patients as being healthy. Moreover, the acquisition of biological data is both difficult and expensive since generating training samples in the biomedical field requires a person with specialized skills and a series of long-term experiments. If synthetic data can be used to supplement and improve real data, more valuable applications can be achieved in different domains with less existing data. Creating synthetic bioinformatic data is a challenging task as the synthetic data should maintain the underlying biological effects.

Kidney diseases, for example, are well-known to be largely multifactorial, having complex and overlapping clinical phenotypes and morphologies, which often result in late diagnosis and chronic progression. Despite advances in computational power and the evolution of machine learning-based methods, the biological complexities that underlay various kidney diseases and the progression towards kidney transplant rejection have continued to make early diagnosis and intervention problematic, especially in resource-inadequate areas. Currently, existing research and applied works have focused on leveraging such methods to better understand multi-organ segmentation and function, where machine learning methods have made certain contributions to more accurate and timely prediction, and better understanding of histologic pathology. However, such methods have been limited in the fields of transplantation and rejection monitoring due to inadequate data availability and have thus yet to break into standard medical practice and diagnostic procedures. With the help of artificial intelligence (AI), it is possible to perform large health screens for potential kidney disease and targeted biomarker and drug discovery thus allowing clinicians to treat patients in a more targeted manner.

Furthermore, AI-assisted diagnostic applications can help shed light on the various etiologies of kidney disease for more precise phenotyping or outcome prediction, thus reducing the possibility of misdiagnosis. The generalization of machine learning models typically relies on the quality of a dataset, as good datasets will enable machine learning classifiers to capture the underlying characteristics efficiently. As a result, machine learning classifiers likely become more robust in generalizing underlying characteristics effectively on unseen data. To achieve a good dataset, the data should be a good representation of the real distribution, and it should cover as many cases as possible with a reasonably large number of samples. However, collecting biomedical data usually requires the involvement of specialized doctors, leading to a high collection cost and data; therefore, it is not always possible to access more patient's data. Thus, creating synthetic datasets is valuable when machine learning algorithms try to learn the underlying characteristics of the data from small imbalanced datasets.

Described herein is a Generative Adversarial Network (GAN) system generated by introducing synthetic data into a biological data set (i.e., data augmentation) to generate synthetic data in a tabular format that, for example, reduces class imbalance when there is an uneven number of samples for all classes present in a dataset. The systems and processes described herein add extraneous synthetic training data into a training set obtained from biological samples to improve the performance of machine learning algorithms and greatly reduce or eliminate biases generated from an uneven number of samples. In some aspects, the systems of the disclosure describe the addition of extraneous synthetic data to a kidney transplant rejection dataset trained primarily on six biomarker features-along with a time feature representing the number of days since an organ transplant (e.g., kidney transplant, pancreas transplant, double kidney plus pancreas transplant) (time post-transplant days: 0 days (surgery day), −1 day (day prior to surgery), +1 day (24 hours post-surgery), etc.) to predict the early failure of a kidney transplant.

In some aspects, the disclosure provides systems generated with different GAN architectures, and the effectiveness of synthetic data generated by GAN-based methods for machine learning algorithms, and processes for utilizing the same. In some aspects, the disclosure describes a comparison of the distribution of first two principal components, and the cumulative sum per feature in a data set comprising only original data collected from biological samples against a synthetic training set having synthetic biomarkers data (i.e., the extraneous data) added therein. In additional aspects, the disclosure describes scores of ROC-AUC, sensitivity, and specificity obtained by machine learning classifiers that are trained with extra synthetic data against classifiers, trained only on the original data. In further aspects, the disclosure describes performances of machine learning classifiers on datasets augmented by one or more GAN architectures described herein, including, but not limited to Conditional Tabular GAN (CTGAN) architectures, statistical oversampling SMOTE architectures, ADASYN architectures, and SVMSMOTE architectures.

The disclosure demonstrates with experimental results that systems and processes utilizing GAN-based data augmentation achieve a significantly greater accuracy when compared to traditional statistical oversampling methods in correctly classifying medical samples. The use of such GAN-based data augmentation approach for medical tabular data provides for a new generation of artificial intelligence applications in the medical field.

### Generative Adversarial Networks for Analysis of Biomarkers

The presence or absence of a biomarker combination in a sample can reflect a status of an organ of the subject. Identification of biomarkers typically involve the use of biochemical assays for identifying “an amount” or a “a level” of the biomarker in a sample. Many assays exist in the art that can be used for the detection of biomarkers in biological samples—e.g., urine or blood-such as genes or protein arrays or metabolite analysis. The use of biochemical assays in this context could requires probing for functional alterations in genes and proteins, the need for a priori knowledge of their function (e.g., antibody detection), as well as extensive assay development and optimization.

With many diseases (e.g., allograft rejection or organ injury), the presence of observable functional biomarkers often occurs late in the disease state. The presence of serum creatinine (sCr) for example, a biomarker commonly used to screen for kidney allograft rejection, is only detected as a late marker of allograft rejection. As such, preventive measures for allograft rejection or kidney injury may be ineffective when developed in connection solely with the detection of a late marker of rejection, such as serum creatinine.

Contributions towards understanding individual biomarkers expressed in allograft rejection, particularly kidney, lung, and heart allograft rejections have been made by methodical evaluation of gene expression data, and “omics” studies. See, e.g., Sigdel T K, Bestard O, Tran T Q, et al. A Computational Gene Expression Score for Predicting Immune Injury in Renal Allografts. PLOS One. 2015; 10 (9): e0138133. Published 2015 Sep. 14.vdoi: 10.1371/journal.pone.0138133; see also Sigdel, Tara, et al. “Assessment of 19 Genes and Validation of CRM Gene Panel for Quantitative Transcriptional Analysis of Molecular Rejection and Inflammation in Archival Kidney Transplant Biopsies.” Frontiers in Medicine, vol. 6, 2019, doi: 10.3389/fmed.2019.00213; see further Sigdel, Tara K., et al. “A Urinary Common Rejection Module (UCRM) Score for Non-Invasive Kidney Transplant Monitoring.” PLOS ONE, vol. 14, no. 7, 2019, doi: 10.1371/journal.pone.0220052. See, also, Khatri, Purvesh, et al. “A Common Rejection Module (CRM) for Acute Rejection across Multiple Organs Identifies Novel Therapeutics for Organ Transplantation.” Journal of Experimental Medicine, vol. 210, no. 11, 2013, pp. 2205-2221., doi: 10.1084/jem.20122709.

Other studies have considered donor derived cell-free DNA (dd-cfDNA) as a potential surrogate biomarker for allograft injury, first in blood, subsequently in urine samples. dd-cfDNA is continually shed into the circulation from the moment the transplanted organ is implanted. One rationale for monitoring dd-cfDNA in transplantation is that cell damage to the allograft leading up to or during episodes of rejection results in release of DNA into the circulation of the recipient and therefore an uptick in dd-cfDNA levels. Thus, due to continual cell turnover, strategies to measure the levels of donor derived cell free DNA (dd-cfDNA) as a surrogate biomarker for allograft injury have been explored as potential surrogate biomarkers for transplant injury (See, e.g., Sarwal and Sigdel WO2014/145232). Such applications, however, are limited by the techniques available for capture of dd-cfDNA.

For instance, some methods for capture/detection of dd-cfDNA required either gender mismatch between donor and recipient or prior genotyping of the donor and recipient. This allows quantification of dd-cfDNA by PCR amplification of the genes found on the Y-chromosome such as the SRY gene. Snyder and colleagues described a universal approach to dd-cfDNA assessment not necessitating gender mismatch (See T. M. Snyder, K. K. Khush, H. A. Valantine, S. R. Quake., Universal noninvasive detection of solid organ transplant rejection. Proc Natl Acad Sci, 108 (2011), pp. 6229-6234). Using genome-wide sequencing of plasma cfDNA in heart transplant recipients, Snyder assessed for SNPs known to be homozygous with different sequences between the donor and recipient and calculated the fraction of dd-cfDNA to total cfDNA. The study found that, with some frequency, the dd-cfDNA levels would rise before the pathologic diagnosis of rejection. However, this approach requires DNA from the donor, which is often impractical, and especially difficult if the transplant was performed years earlier.

An improvement on these technologies required the use of targeted next generation sequencing (NGS) techniques to quantify dd-cfDNA without the need for prior genotyping of the donor and recipient. These NGS assays include AlloSure® (CareDx, Inc., Brisbane CA) and Prospera® (Natera, Inc., San Carlos CA). Allosure® has been analytically validated in a Clinical Laboratory Improvement Amendments (CLIA) setting. Prospera® (Natera, Inc., San Carlos CA) was adapted for use in kidney transplantation from an approach developed for non-invasive prenatal testing (NIPT). Nevertheless, both approaches require NGS sequencing of samples making these products costly for continuous monitoring, and often impractical.

Sarwal and colleagues investigate uses of various samples, including urine, as non-invasive sources of other informative biomarkers for the monitoring of different types of solid organ transplants (See, e.g., U.S. Pat. Nos. 10,982,272; 10,995,368; 11,124,824; and U.S. patent application Ser. Nos. 17/376,919 and 17/498,489). Sarwal recognized that Alu elements are the most abundant transposable elements in the human genome, containing over one million copies dispersed throughout the human genome. Recognizing the abundance of ALU repeats, Sarwal created a ratio of ALU repeats in a urine sample of a transplant patient over the number of ALU repeats in a urine sample from a normal population. The ratio could be used as a proxy of injury, however, on its own it was not sufficiently informative.

Additional studies have begun to explore potential combinations of biomarkers as proxies for allograft injury. For instance, QSant™ utilizes a composite score of various biomarkers of distinct biochemical characteristics, i.e., proteins, metabolites, and nucleic acids. (See Yang, Sarwal, et al., A urine score for noninvasive accurate diagnosis and prediction of kidney transplant rejection. Science Translational Medicine, 18-3-2020, Vol. 12, Issue 535). Yang et al. demonstrated that a urinary composite score of six biomarkers—an inflammation biomarker (e.g., CXCL-10, also known as IP-10); an apoptosis biomarker (e.g., clusterin); a cfDNA biomarkers; a DNA methylation biomarker; a creatinine biomarker; and total protein—enables diagnosis of Acute Rejection (AR), with a receiver-operator characteristic curve area under the curve of 0.99 and an accuracy of 96%. Notably, QSant™ (formerly known as QiSant™) predicts acute rejection before a rise in a stand-alone serum creatinine test, enabling earlier detection of rejection than currently possible by current standard of care tests.

However, the analysis of the data obtained in such studies can be challenging in part because many biological datasets available for these studies arise from imbalanced datasets; datasets where there is an uneven number of samples for all classes present in a dataset. This can cause machine learning algorithms to produce a poor performance on the minority classes while favoring bias towards the majority class. The disclosure contemplates a scenario where synthetic data is used to supplement and improve real data obtained in such studies to reduce class imbalance and achieve more valuable applications in different domains with less existing data.

### Generative Adversarial Networks

Creating synthetic datasets is valuable when machine learning algorithms try to learn the underlying characteristics of the data from small imbalanced datasets. Machine-learning algorithms find and apply patterns in data. Multivariate machine learning, linear and nonlinear fitting algorithms can also be applied in biomarker searches. Machine learning is generally supervised or unsupervised. In supervised learning, the most prevalent, the data is labeled to tell the machine exactly what patterns it should look for. For instance, samples of a patient with a known diagnosis of acute rejection are labeled as “acute rejection.” Samples from “normal” patients are labeled “stable.” The algorithm then starts looking for patterns that are clearly distinct between “normal” and “acute rejection.” In unsupervised learning, the data has no labels. The machine algorithm looks for whatever patterns it can find. This can be interesting if, for instance, every sample analyzed is from a subject who received an allograft. It could, for example, be used for detection of a broad allograft specific marker.

The generalization of machine learning models relies on the quality of a dataset as good datasets will enable machine learning classifiers to capture the underlying characteristics well. As a result, machine learning classifiers will become more robust in generalizing underlying characteristics effectively on unseen data. To achieve a good dataset, the data generally should be a good representation of the real distribution, and it should cover as many cases as possible with a reasonably large number of samples. However, collecting biomedical data usually requires the involvement of specialized doctors, leading to a high collection cost and data; therefore, it is not always possible to access more patient's data. Another reason for creating synthetic data is to avoid using the original data to train machine models for privacy reasons. For instance, medical samples consisting of sensitive personal information about patients such as weight, height, and date of birth should be strictly protected for privacy reasons since working directly with such information could jeopardize its security. The present disclosure addresses these challenges by a) generating a synthetic dataset that augments input in biological samples by providing synthetic (i.e., extraneous training features to an original data; and b) training machine learning models on the generated synthetic dataset without training on original data.

While there has been an explosion of biomarker discovery efforts utilizing genomics, proteomics and metabolomics, these technologies also focus on the characterization of biomarkers present in original biological samples. Biological samples can particularly benefit from synthetic data augmentation technology, in part because of challenges obtaining sufficient quantities of original samples or because of challenges preserving the integrity of all biomarkers in an original biological sample that become features in a machine learning model. The present disclosure demonstrates the utility of synthetic data augmentation technology in biological samples and demonstrates its utility in a particular embodiment of a kidney transplant rejection dataset consisting of six biomarkers; namely cell-free DNA (cfDNA), methylated cell-free DNA (m-cfDNA), at least one inflammation marker(s), at least one apoptosis marker(s), total protein, and creatinine for predicting the early failure of kidney transplant. The biological roles of these biomarkers for the assessment of kidney injury and acute rejection in patients can have a turnaround time of less than 3 days and have demonstrated efficiency in supporting critical patient management decisions. See, e.g., U.S. Pat. Nos. 10,982,272 and 10,995,368. See also; A urine score for noninvasive accurate diagnosis and prediction of kidney transplant rejection, Science Translational Medicine 18-3-2020: Vol. 12, Issue 535, eaba2501. Following kidney transplants, it is essential to monitor subjects for evidence of rejection to reduce the risk of graft loss. In this disclosure, the performance of machine learning algorithms was demonstrated to improve when algorithms trained on datasets obtained from urine samples of subjects (i.e., real training data) were combined with synthetic data generated by the GAN-based data augmentation methods.

### Generative Adversarial Networks for Analysis of Urine Biomarkers

In one aspect, the instant disclosure provides a synthetic data augmentation approach for medical tabular data that improves the analysis of combinations of biomarkers that can be used for high accuracy monitoring of the integrity of a solid organ allograft after a transplant. The present disclosure describes such an analysis in a kidney transplant rejection dataset that consists of six biomarkers named cell-free DNA (cfDNA), methylated cell-free DNA (m-cfDNA), CXCL10, clusterin, total protein, and creatinine, for predicting the early failure of kidney transplant.

Kidney disease is an important medical and public health burden globally, with both AKI and CKD bringing about high morbidity and mortality, as well as contributing to huge healthcare costs. Due to the high heterogeneity in disease manifestation, progression, and treatment response, the present disclosure considered leveraging novel big-data and AI methods to solve the challenges that come with dealing with these complex diseases, and disease-related injury. The present disclosure considered Generative Adversarial Networks (GANs), first introduced in 2014 by Goodfellow et al, and significantly improved the foundational approach to provide new opportunities to solve data scarcity problems, helping powerful machine learning applications overcome the barrier of small biological sample sizes, particular sample sizes with uneven distribution.

GANs provide a strategy of training a generative model that automatically discovers and learns patterns based on deep neural networks, consisting of the generator network and discriminator network. The generator's role is to generate new plausible examples from the problem domain, and the discriminator's role is to classify examples as either real (from the domain) or fake (e.g., synthetic, or generated). The two neural networks learn simultaneously from training data in an adversarial zero-sum game fashion where one neural network's loss is the gain of another.

In the present disclosure we demonstrate that the GAN-based data augmentation methods can be applied to generate quality synthetic samples that resemble the original distribution of the real-world data it is provided. More importantly though, the above works and related research demonstrate that GAN-based data generation methods can recapitulate the biological complexity seen in the various kinds of genetic, proteomic, and cell-type data often analyzed in diagnostic and therapeutic research. The present disclosure demonstrates that the systems, processes, and methods disclosed herein can be successfully applied to biological data in various medical fields; thus demonstrating that GAN-powered generative models can be a valuable tool to generate synthetic biomarkers data for biological samples for more robust analyses.

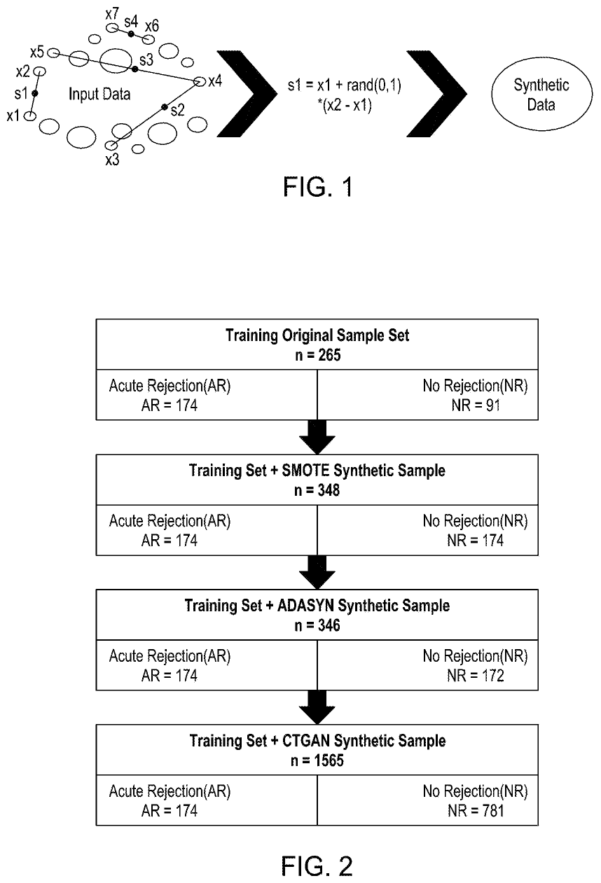

In order to address small sample size problems, several oversampling methods have been proposed in previous studies. The present disclosure provides the use of GAN-based synthetic data technology can be a more effective strategy than previous oversampling methods to overcome issues with imbalanced datasets. In some aspects, the present disclosure contemplates and implements oversampling methods, including random oversampling in its analysis. FIG. 1 (FIG. 1) illustrates a traditional oversampling method (SMOTE). As shown in FIG. 1, the input data (majority class samples are larger circles; minority class samples are smaller circles) is processed with SMOTE methodology (minority oversampling) for synthetic data calculation which then produces the synthetic data.

In some aspects, the present disclosure contemplates a use of Synthetic Minority Oversampling Technique (SMOTE), Borderline-SMOTE, Borderline Oversampling with SVM, and Adaptive Synthetic Sampling (ADASYN), and other suitable methodologies for the analysis of biomarkers in a biological samples (e.g., blood or urine).

In some aspects, an exemplary oversampling method considered in the present disclosure comprises randomly duplicating training examples of the minority class (i.e., Random Oversampling).

In some aspects, an exemplary oversampling method considered in the present disclosure comprises Synthetic Minority Oversampling Technique (SMOTE), which works by selecting examples that are close in the feature space, drawing a line between the samples in the feature space and drawing a new sample as a point along the line.

In yet other aspects, an exemplary oversampling method considered in the present disclosure comprises novel minority oversampling techniques that consider k-nearest neighbor classification models and only generated the minority synthetic samples near the borderline. SMOTE-SVM oversampling method is an extension to SMOTE that fits a support vector machine algorithm to the dataset and uses the decision boundary defined by support vectors to generate synthetic samples.

In another aspects, an exemplary oversampling method considered in the present disclosure comprises an adaptive synthetic sampling approach, which utilizes a weighted distribution for minority class and generates synthetic samples inversely proportional to the density of the examples in the minority class.

In another aspect, the disclosure contemplates a majority weighted minority oversampling technique, whose method aimed to generate more selected synthetic minority class samples by assigning weights based on their Euclidian distance from the nearest majority class instance.

Other methods have been developed to meet dataset demands, and the present disclosure contemplates alternative suitable methods for imbalance learning for machine learning algorithms by rebalancing the class distribution for an imbalanced dataset.

### Other Definitions

For purposes of interpreting this specification, the following definitions will apply and whenever appropriate, terms used in the singular will also include the plural and vice versa.

### Samples

The terms “biological sample” or “sample” as used herein, refers to a mixture of cells, tissue, and liquids obtained or derived from an individual that contains a cellular and/or other molecular entity that is to be characterized and/or identified, for example based on physical, biochemical, chemical and/or physiological characteristics. In one embodiment the sample is liquid (i.e., a biofluid), such as urine, blood, serum, plasma, saliva, phlegm, etc. In other embodiments, the sample is a histological section, such as a solid tissue section from a biopsy.

### Subjects

A subject can be any human or animal, collectively “individuals”, that has received an allograft. For instance, subjects can be humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants. In specific cases, a subject is a pediatric recipient of an allograft.

A “subject”, also referred to as an “individual” can be a “patient.” A “patient,” refers to a subject who is under the care of a treating physician. In one embodiment, the patient is suffering from renal damage or renal injury. In another embodiment, the patient is suffering from renal disease or disorder. In another embodiment, the patient has had a renal transplant and is undergoing of renal graft rejection. In yet other embodiments, the patient has been diagnosed with renal injury, renal disease, or renal graft rejection, but has not had any treatment to address the diagnosis.

### Probes

“Hybridization”, “probe hybridization”, “cfDNA probe hybridization”, or “Alu probe hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the pairing with a cfDNA sequence (e.g., probe hybridization to an Alu region of a cfDNA), initiation of PCR, or the cleavage of a polynucleotide by an enzyme. A sequence capable of hybridizing with a given sequence is referred to as the “complement” of the given sequence.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

As used herein the term “amino acid” includes natural and/or unnatural or synthetic amino acids, including glycine and both the D or L optical isomers, and amino acid analogs and peptidomimetics.

As used herein the term metabolite refers to intermediate or end products of metabolism. The term metabolite is usually used for small molecules, but it can also include amino acids, vitamins, nucleotides, antioxidants, organic acids, and vitamins.

As used herein, the term “domain” or “protein domain” refers to a part of a protein sequence that may exist and function independently of the rest of the protein chain.

As used herein, the terms “disorder” or “disease” and “injury” or “damage” are used interchangeably. It refers to any alteration in the state of the body or one of its organs and/or tissues, interrupting or disturbing the performance of organ function and/or tissue function (e.g., causes organ dysfunction) and/or causing a symptom such as discomfort, dysfunction, distress, or even death to a subject afflicted with the disease.

A subject “at risk” of developing renal injury, renal disease or renal graft rejection may or may not have detectable disease or symptoms and may or may not have displayed detectable disease or symptoms of disease prior to the treatment methods described herein. “At risk” denotes that a subject has one or more risk factors, which are measurable parameters that correlate with development of renal injury, renal disease, or renal graft rejection, as described herein and known in the art. A subject having one or more of these risk factors has a higher probability of developing renal injury, renal disease, or renal graft rejection than a subject without one or more of these risk factor(s).

The term “condition” is used herein to refer to the identification or classification of a medical or pathological state, disease, or diagnosis. For example, “condition” may refer to a healthy condition of subject, a stable condition of a subject who received an allograft, or it may refer to identification of a disease. A disease can be renal injury, renal disease (e.g., CKI or AKI), or renal graft rejection. “Diagnosis” may also refer to the classification of a severity of the renal injury, renal disease, or renal graft rejection. Diagnosis of the renal injury, renal disease, or renal graft rejection may be made according to any protocol that one of skill of art (e.g., a nephrologist) would use.

The term “companion diagnostic” is used herein to refer to methods that assist in making a clinical determination regarding the presence, degree or other nature, of a particular type of symptom or condition of renal injury, renal disease, or renal graft rejection. For example, a companion diagnostic of renal injury, renal disease, or renal graft rejection can include measuring the fragment size of cell free DNA.

The term “prognosis” is used herein to refer to the prediction of the likelihood of the development and/or recurrence of an injury being treated with an allograft, e.g., a renal injury, renal disease, or renal graft rejection. The predictive methods of the invention can be used clinically to make treatment decisions by choosing the most appropriate treatment modalities for any particular patient. The predictive methods of the present invention are valuable tools in predicting if and/or aiding in the diagnosis as to whether a patient is likely to develop renal injury, renal disease, or renal graft rejection, have recurrence of renal injury, renal disease, or renal graft rejection, and/or worsening of renal injury, renal disease, or renal graft rejection symptoms.

“Treating” and “treatment” refers to clinical intervention in an attempt to alter the natural course of the individual and can be performed before, during, or after the course of clinical diagnosis or prognosis. Desirable effects of treatment include preventing the occurrence or recurrence of renal injury, renal disease, or renal graft rejection or a condition or symptom thereof, alleviating a condition or symptom of renal injury, renal disease, or renal graft rejection, diminishing any direct or indirect pathological consequences of renal injury, renal disease, or renal graft rejection, decreasing the rate of renal injury, renal disease, or renal graft rejection progression or severity, and/or ameliorating or palliating the renal injury, renal disease, or renal graft rejection. In some embodiments, methods and compositions of the invention are used on patient sub-populations identified to be at risk of developing renal injury, renal disease, or renal graft rejection. In some cases, the methods and compositions of the invention are useful in attempts to delay development of renal injury, renal disease, or renal graft rejection. Beneficial or desired clinical results are known or can be readily obtained by one skilled in the art. For example, beneficial or desired clinical results can include, but are not limited to, one or more of the following: monitoring of renal injury, detection of renal injury, identifying type of renal injury, helping renal transplant physicians to decide whether or not to send transplant patients to go for a biopsy and make decisions for the purposes of clinical management and therapeutic intervention.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from what occurs in nature. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related or are only partially structurally related. An “orthologue” of a protein as used herein is a protein of a different species which performs the same or a similar function as the protein it is an orthologue of Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22 (4): 359-66. doi: 10.1002/pro.2225).

### EXAMPLES

### Example 1: Generative Adversarial Networks for Generating Synthetic Biomarkers Data for Urine Samples

### Data Collection.

The study included 379 independent biopsy matched urine samples obtained with informed consent from 309 pediatric (3 to 18 years of age) and adult recipients (18 to 76 years of age) of renal allografts, transplanted at three different transplant centers (University of California San Francisco (UCSF), San Francisco, USA), Stanford University (Palo Alto, CA) and Instituto Nacional de Ciencias Medicas y Nutricion (Mexico City, Mexico).

Of the 379 samples, acute kidney allograft rejection (AR) was confirmed by the paired biopsy read in 243 samples and a no-rejection or stable (STA) phenotype confirmed in 136 samples. Urine samples were collected from these patients from 1 to 1539 days post-transplant. Custom generated ELISAs for m-cfDNA, CXCL10, and clusterin concentration were used for these biomarkers. cfDNA was detected with a prove as described by Sarwal and colleagues (See, e.g., U.S. Pat. Nos. 10,982,272; 10,995,368; 11,124,824; and U.S. patent application Ser. Nos. 17/376,919 and 17/498,489). Both DNA assays used SuperSignal ELISA for luminescent detection. Analyte concentrations from the 379 independent biological samples with corresponding biomarker data (cfDNA, m-cfDNA, CXCL10, clusterin, creatinine, and total protein were measured.

Synthetic urine samples were generated from a learned distribution of urinary analyte concentrations based on real biological samples with corresponding biomarker data (cfDNA, m-cfDNA, CXCL10, clusterin, creatinine, and total protein).

After randomly split of the original data into a 70% training set and 30% test set, there were 174 biopsy confirmed acute kidney allograft rejection (AR) phenotype and 91 no-rejection (NR) or stable (STA) phenotype in the training set. In the test set, there were 69 biopsy confirmed acute kidney allograft rejection (AR) phenotype and 45 no-rejection or stable (STA) phenotype. The following schemes were used to enlarge the training dataset with different data augmentation methods.

FIG. 2 (FIG. 2) is a schematic of various GANS strategies utilized on the aforementioned datasets to test the process for enlarging the dataset with different data augmentation methods. As depicted on FIG. 2 an original sample set of 295 inputs (Acute Rejection (AR)=174; No Rejection (NR)=91 was used for training.

Subsequently, Synthetic Minority Oversampling Technique (SMOTE) was used as a statistical technique for increasing the number of cases in the dataset in a balanced way. The module worked by generating new instances from existing minority cases (NR=91) that were supplied as input. This implementation of SMOTE did not change the number of majority cases. Further, the new synthetic data were not just copies of existing minority cases. Instead, the algorithm took samples of the feature space for each target class and its nearest neighbors to create a balanced sample with AR=174, NR=174 for a total of n=348.

In parallel, adaptive synthetic sampling approach for imbalanced learning (ADASYN) methodology was used to generate the synthetic data points required to balance the dataset. The major difference between SMOTE and ADASYN is the difference in the generation of synthetic sample points for minority data points. In ADASYN, we considered a density distribution rx which thereby decided the number of synthetic samples to be generated for a particular point, whereas in SMOTE, there was a uniform weight for all minority points. This strategy created a balanced sample with AR=174, NR=172 for a total of n=346, as illustrated in FIG. 2.

In parallel, CTGAN a collection of deep learning based synthetic data generators for single table data. CTGAN (for “conditional tabular generative adversarial networks”) used GANs to build and perfect synthetic data tables. GANs are pairs of neural networks that creates a first row of synthetic data—and the second, called the discriminator, tries to tell if it's real or not. Eventually, the generator can generate synthetic data which the discriminator cannot distinguish from real data. This strategy created a balanced sample with AR=784, NR=784 for a total of n=1,565, as illustrated in FIG. 2.

### Example 2: Creating Machine Learning Classifiers with Various GANs′

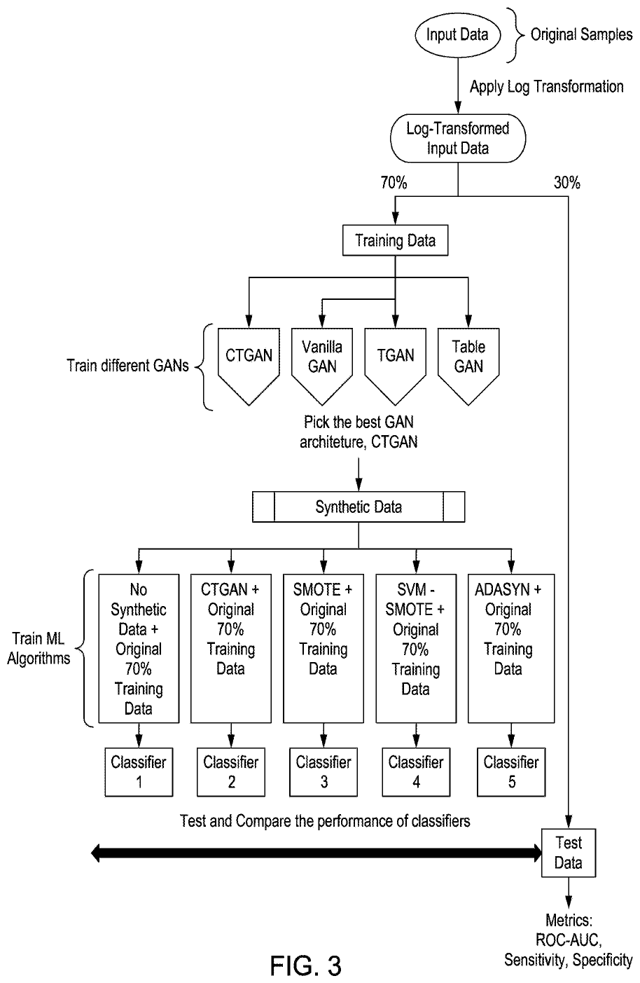

Synthetic urine samples were generated from a learned distribution of urinary analyte concentrations based on real biological samples with corresponding biomarker data (cfDNA, m-cfDNA, CXCL10, clusterin, creatinine, and total protein). FIG. 3 (FIG. 3) illustrates the strategy for training different Generative Adversarial Networks (GANs); incorporating extraneous data (i.e., synthetic samples or synthetic features or extraneous data) therein, and subsequently training different algorithms outlined in this example.

In order to develop and train different GANs, the data was split into training and test sets using a random 70/30 split, respectively, and four different GANs were performed: CTGAN (conditional tabular generative adversarial networks), Vanilla GAN, Tabular GAN (TGAN) and Table GAN. See FIG. 3; “train different GANs”.

Log transformation was applied to the data to transform the skewed distribution of the aforementioned biomarkers and to help reduce the ranges of values that the generator must produce. Models were subsequently trained with both an identified and an unidentified target variable to generate high quality synthetic minority samples.

TGAN is a tabular data synthesizer that uses an LSTM to generate synthetic data column by column, and each column depends on the previously generate columns. When generating a column, the attention mechanism of TGAN pays attention to previous columns that are highly related to the current column.

Table GAN uses convolutional networks in both the generator and the discriminator. When tabular data contains a label column, a prediction loss is added to the generator to explicitly improve the correlation between the label column and other columns.

Vanilla GAN uses a minmax algorithm, including a discriminator and generator with 4 dense layers in its architecture, optimizing binary cross entropy loss function, which computes log loss of both generator and discriminator predicted probabilities. Conditional

Tabular GAN is a GAN-based data augmentation method to handle challenges in tabular data generation tasks such as non-Gaussian, multimodal distribution, and the imbalanced discrete columns that previous statistical and deep neural network methods fail to address.

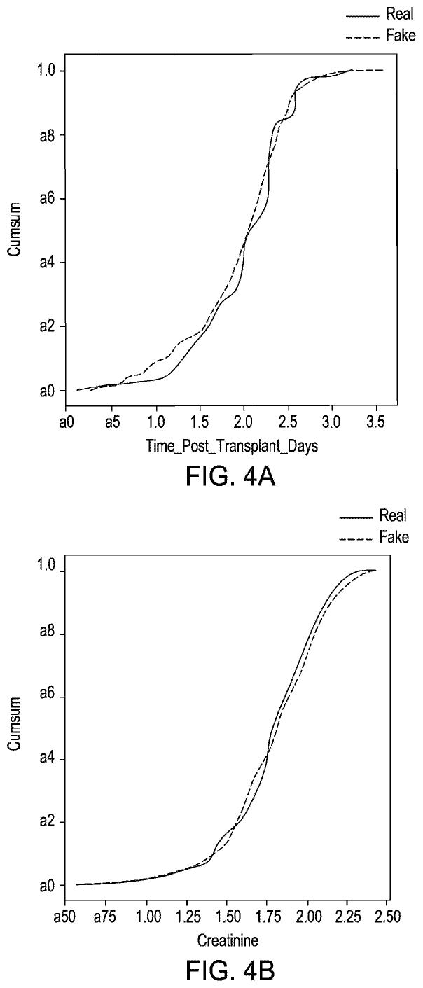

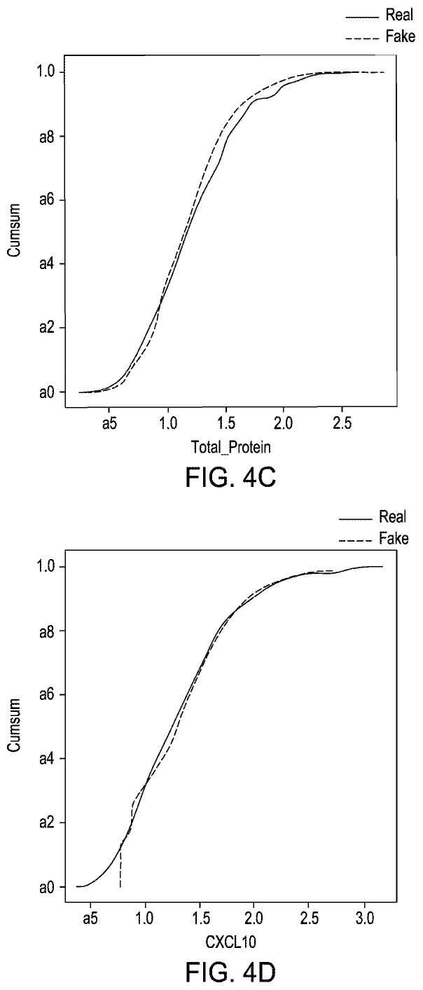

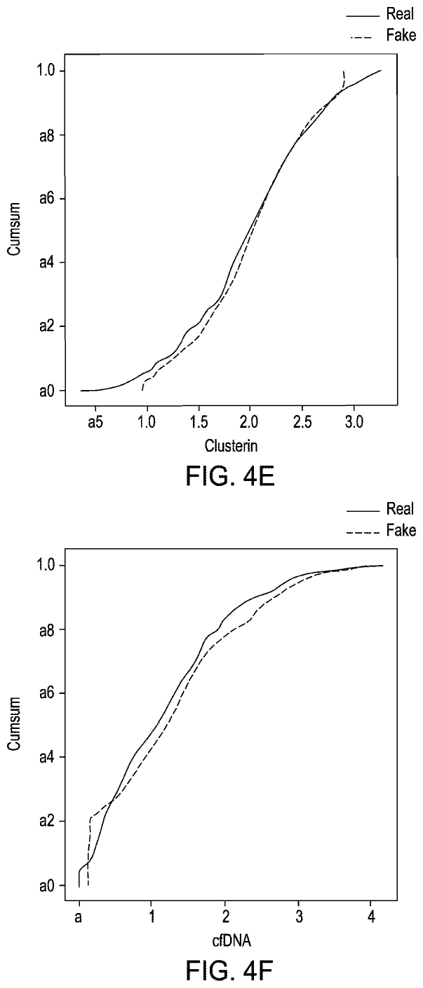

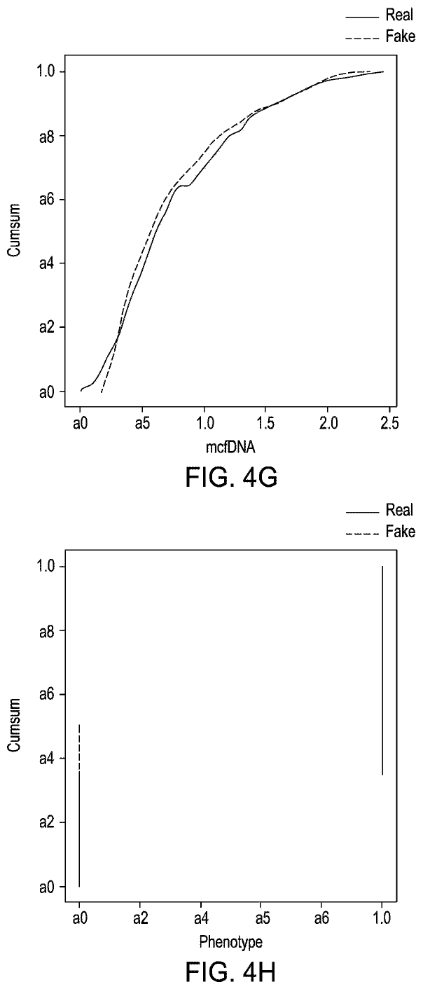

FIG. 4A-FIG. 4H (FIGS. 4A-4H) collective illustrate a comparison between a range of time points and exemplary biomarkers measured with original biological samples (i.e., features on original biological samples) and synthetic samples (i.e., synthetic features) based on their distribution produced by CTGAN (conditional tabular generative adversarial networks). FIG. 4A illustrates a comparison between original samples and synthetic samples (i.e., synthetic features) based on cumulative sums per feature of 6 biological features produced by CTGAN over a period of time after transplant. FIGS. 4B-4G illustrate a comparison between original samples and synthetic samples (i.e., synthetic features) based on each individual biological feature used on an exemplary test, namely the QSant™ diagnostic test for allograft rejection. FIG. 4B illustrate performance of a creatinine biomarker, FIG. 4C illustrate the performance of a total protein biomarker, FIG. 4D illustrate the performance of an exemplary inflammatory biomarker, FIG. 4E illustrate the performance of an exemplary clusterin biomarker, FIG. 4F illustrate the performance of an exemplary cfDNA biomarker. FIG. 4H illustrate the distribution of real vs fake phenotype.

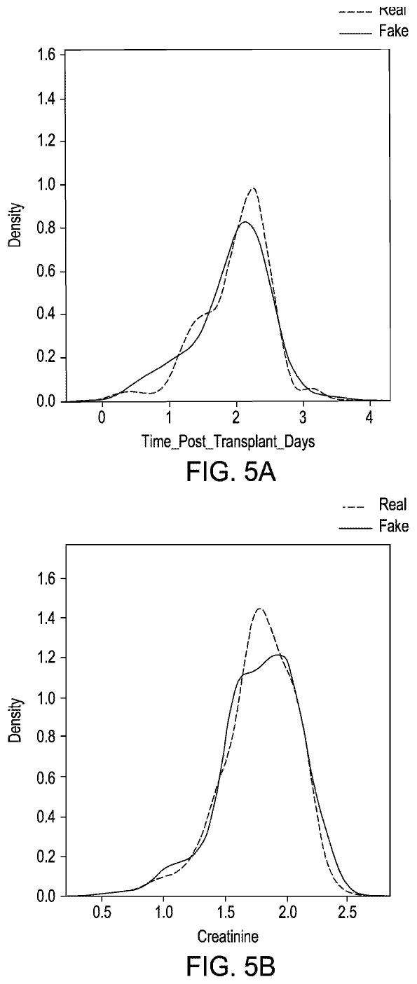

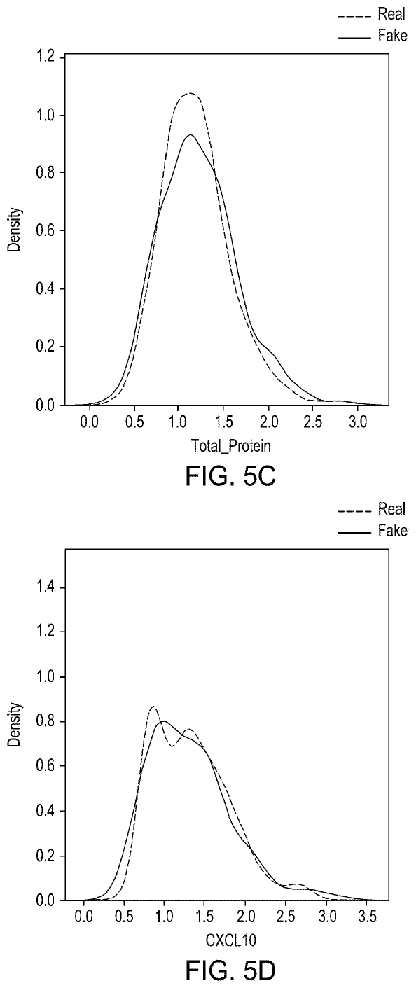

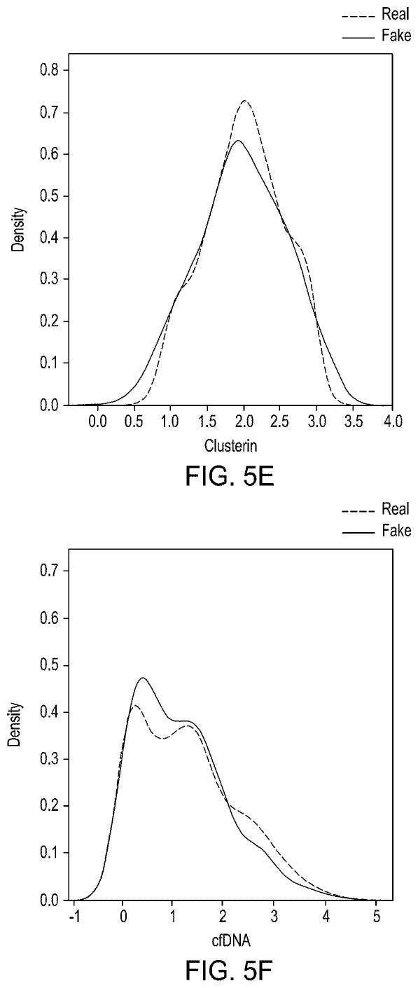

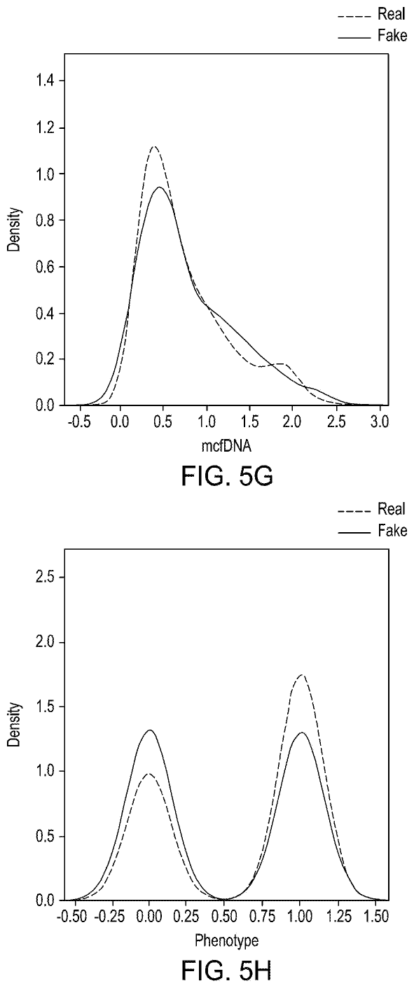

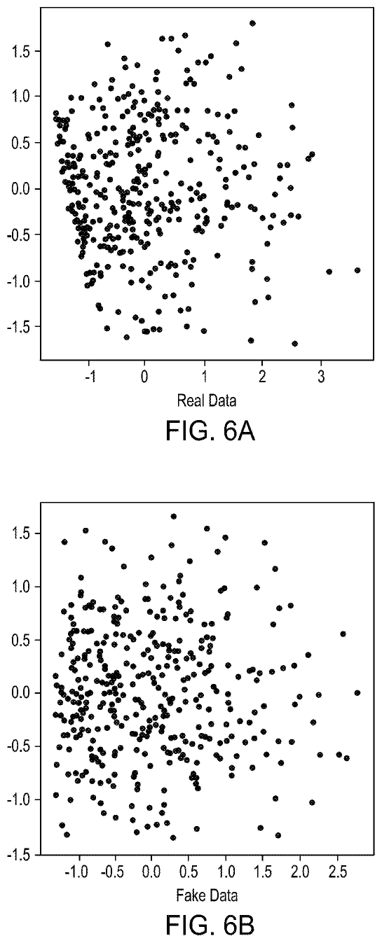

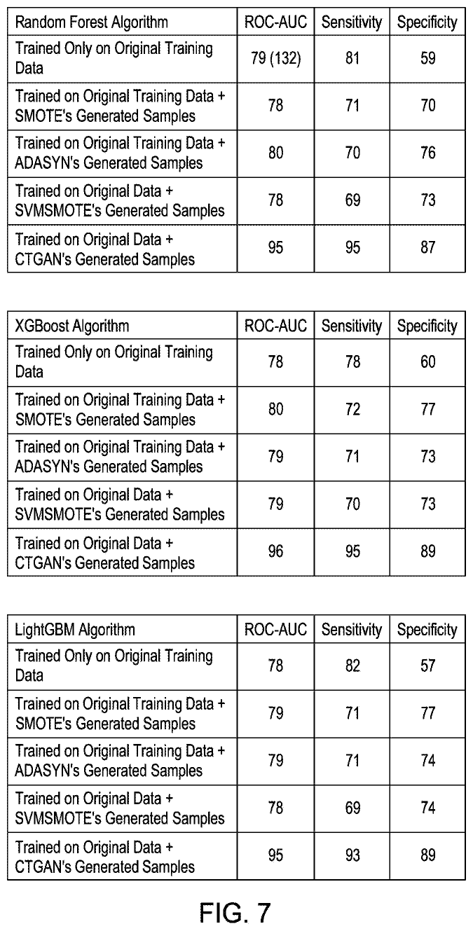

FIG. 5A-FIG. 5H (FIGS. 5A-5H) collectively illustrate a comparison between a range of time points and exemplary biomarkers measured with original biological samples (i.e., features on original biological samples) and synthetic samples (i.e., synthetic features) based on the first two principal components produced by CTGAN. FIGS. 5B-5G illustrate a comparison between original samples and synthetic samples (i.e., synthetic features) based on each individual biological feature used on an exemplary test, namely the QSant™ diagnostic test for allograft rejection. FIG. 5B illustrate performance of a creatinine biomarker, FIG. 5C illustrate the performance of a total protein biomarker, FIG. 5D illustrate the performance of an exemplary inflammatory biomarker, FIG. 5E illustrate the performance of an exemplary clusterin biomarker, FIG. 5F illustrate the performance of an exemplary cfDNA biomarker. FIG. 5H illustrate the phenotype. FIG. 6A-FIG. 6B (FIGS. 6A-6B) collectively illustrate the result analysis of machine learning algorithms' performance on training samples+synthetic samples augmented by different oversampling techniques.

It was observed that training CTGAN without class labels generated provides a realistic synthetic data for biomarker values (with high sensitivity and high specificity) as compared to other GAN architectures based on their distributions, cumulative sum per feature, and the first two principal components. Machine learning classifiers were then built on the training set merged with synthetic samples, and performances of the classifiers oversampled by CTGAN were compared against traditional oversampling methods such as SMOTE, SVM_SMOTE, ADASYN, and a baseline of non-oversampled data.

But more importantly, the data suggests that a variety of distinct methods can be used to generate synthetic data that closely tracks the performance of the biological data. Based on the aforementioned data, various systems can be configured to balance an imbalanced dataset obtained from a biological sample, these samples can train the data with CTGAN, Vanilla GAN, TGAN, and Table GAN strategies to produce synthetic data. Such synthetic data can then be used to train various ML algorithms, including CTGAN, SMOTE, SVM-SMOTE, ADASYN machine learning algorithms.

The present disclosure contemplates that such strategies can be used with biological samples obtained from urine as described in the examples, but also from blood, serum, plasma, bronchioalveolar fluid, or another suitable source of a biological material.

### Example 3: Synthetic Urine Samples Generated with Conditional Tabular Generative Adversarial Network (CTGAN)

The Conditional Tabular Generative Adversarial Network (CTGAN) with Wasserstein loss (W-loss) and gradient penalty were used as an illustrative GAN architecture to generate final synthetic urine samples in this example. In contrast to min-max normalization that previous models used to manage complicated distributions, CTGAN introduced new techniques such as a conditional generator and training-by-sampling to manage imbalanced discrete columns and mode-specific normalization. The training process of the traditional GAN was a minimax game using binary cross-entropy loss (Bce-loss); however, the training of GAN with Bce_loss was prone to mode collapse and vanishing gradient problems, especially when generated examples were vastly different from real examples. Mode collapse happens when the generator learns to fool the discriminator by producing examples from a single class from the whole training dataset like handwritten number ones, collapsing to single-mode or the whole distribution of possible handwritten digits. Real-world datasets may have many modes related to each possible class within them such as the digits in the dataset of handwritten digits.

To solve this mode collapse and vanishing gradient descents, the present disclosure used CTGAN applying the Wasserstein loss (W-loss) function, including gradient penalty regularization term along with a critic network/discriminator that tries to maximize the distance between the real distribution and the fake distribution, approximating Earth Mover Distance, i.e., the amount of effort it takes to make the generated distribution equal to the real distribution. W-loss can be expressed as

$${min}_{g} {max}_{c} 𝔼 ( c ( x ) ) - 𝔼 ( c ( g ( z ) ) ) ,$$

and Bce-loss can be expressed as

$${min}_{d} {max}_{g} [ 𝔼 ( log ( d ( x ) ) ) + 𝔼 ( 1 - log ( d ( g ( z ) ) ) ) ] ;$$

where E(x)=expected value of function (x), Log (x)=logarithmic value of function (x), d(x)=performance of discriminator for real observations, d(g(z))=performances of discriminator for fake observations produced by generator, c(x)=critic function of a real observations, c(g(z))=critic function of fake observations. As W-loss does not require to have a sigmoid activation function in the output layer; the gradient of this loss function will not approach zero. This is enforced by the 1-Lipschitz Continuous condition, which utilizes a regularization term with gradient penalty for W-loss, allowing improved discrimination of real vs. fake observations, without degrading discriminator feedback back to the generator.

The generator will thus provide useful feedback back from the critic, which prevents mode collapse in vanishing gradient problems. In other words, the 1-Lipschitz Continuous condition helps the training of the GAN maintain greater stability by assuring that W-loss function is not only continuous and differentiable at every single value. W-Loss with 1-Lipschitz Continuity condition can be expressed as

$${min}_{g} {max}_{c} 𝔼 ( c ( x ) ) - 𝔼 ( c ( g ( z ) ) ) + λ reg ,$$

where λreg=regularization of the critic's gradient.

### Example 4: Result Analysis of Machine Learning Algorithms' Performance on Training Samples+Synthetic Samples Augmented by Different Oversampling Techniques

The disclosed experiment aimed to achieve the following analysis:
i) to understand if GAN-based data augmentation methods could be utilized to generate high-quality synthetic urine samples,
ii) to understand whether such methods can be outperform traditional oversampling methods for improving the quality of biomarkers data, and
iii) to conclude whether GAN can provide an opportunity to improve the performance of supervised machine learning classifiers in a small imbalanced dataset for predicting the failure of kidney transplant rejections.

Table GAN, Vanilla GAN, TGAN, and CTGAN models were run and tested in their ability to build high-quality synthetic data. Results demonstrated that the disclosed GAN methods performed best in generating synthetic data that closely matched the biopsy data; with the CTGAN model outperforming other architectures in generating synthetic data. The CTGAN model was thus chosen for further analysis.

### CTGAN Analysis

From 265 samples in our training set, CTGAN was used to generate 1300 synthetic urine samples for additional training samples. Machine learning classifiers such as the Random Forest Classifier, Xgboost Classifier, and LightGBM Classifier were then implemented to determine whether at least the disclosed machine learning classifiers could benefit from adding extra synthetic training data into a real training set.

We compared the performances of the classifiers oversampled with Conditional Tabular GAN, SMOTE, SVMSMOTE, and ADASYN, including non-oversampled data as a baseline data. We trained all the classifiers with selected hyperparameters based on a comprehensive hyperparameter grid search performed on a new training set, which consisted of 30% synthetic samples and 70% of original samples. These classifiers were then tested on the 30% test set of the original dataset (n=114), and the performances of the classifiers were measured based on roc-auc, sensitivity, and specificity metrics. Based on the results from FIG. 7, machine learning classifiers perform well when high-quality synthetic training data is added to augment biological data, where GAN-based data augmentation methods in particular helped all three machine learning classifiers more accurately predict acute rejection in renal transplantation over and above other oversampling techniques.

We also analyzed the feature importance of the Random Forest Classifier after training, and the feature importance confirmed that key biomarkers from a biological perspective still appear to make the most contributions in the algorithm. Thus, the potential use of this technique to create synthetic data in scenarios with small imbalanced datasets provides a valuable solution for machine learning applications in the biomedical field.

Table 1—is a tabulation of the performances of Machine Learning Algorithms on the disclosed Kidney Transplant Rejection Dataset of Example 1 with synthetic urine samples.

TABLE 1

| Random Forest | | | |
|---|---|---|---|
| Algorithm | ROC-AUC | Sensitivity | Specificity |
| Trained Only on | 79 | 81 | 59 |
| Original Training | (132) | | |
| Data | | | |
| Trained on Original | 78 | 71 | 70 |
| Training Data + | | | |
| SMOTE's Generated | | | |
| Samples | | | |
| Trained on Original | 80 | 70 | 76 |
| Training Data + | | | |
| ADASYN's Generated | | | |
| Samples | | | |
| Trained on Original | 78 | 69 | 73 |
| Data + SVMSMOTE's | | | |
| Generated Samples | | | |
| Trained on Original | 95 | 95 | 87 |
| Data + CTGAN's | | | |
| Generated Samples | | | |

TABLE 2

| XGBoost | | | |
|---|---|---|---|
| Algorithm | ROC-AUC | Sensitivity | Specificity |
| Trained Only on | 78 | 78 | 60 |
| Original Training | | | |
| Data | | | |
| Trained on Original | 80 | 72 | 77 |
| Training Data + | | | |
| SMOTE's Generated | | | |
| Samples | | | |
| Trained on Original | 79 | 71 | 73 |
| Training Data + | | | |
| ADASYN's | | | |
| Generated Samples | | | |
| Trained on Original | 79 | 70 | 73 |
| Data + SVMSMOTE's | | | |
| Generated Samples | | | |
| Trained on Original | 96 | 95 | 89 |
| Data + CTGAN's | | | |
| Generated Samples | | | |

| LightGBM Algorithm | ROC-AUC | Sensitivity | Specificity |
|---|---|---|---|
| Trained Only on | 78 | 82 | 57 |
| Original Training | | | |
| Data | | | |
| Trained on Original | 79 | 71 | 77 |
| Training Data + | | | |
| SMOTE's Generated | | | |
| Samples | | | |
| Trained on Original | 79 | 71 | 74 |
| Training Data + | | | |
| ADASYN's Generated | | | |
| Samples | | | |
| Trained on Original | 78 | 69 | 74 |
| Data + SVMSMOTE's | | | |
| Generated Samples | | | |
| Trained on Original | 95 | 93 | 89 |
| Data + CTGAN's | | | |
| Generated Samples | | | |

Our experiments showed the potential use of Generative Adversarial Network-based data augmentation methods to create synthetic urine samples in scenarios with a small imbalanced biomedical dataset for machine learning systems. By comparing GAN-based data augmentation methods with traditional statistical sampling techniques, we verified that GAN-based techniques can model complicated distributions of tabular data for more robust results of machine learning algorithms.

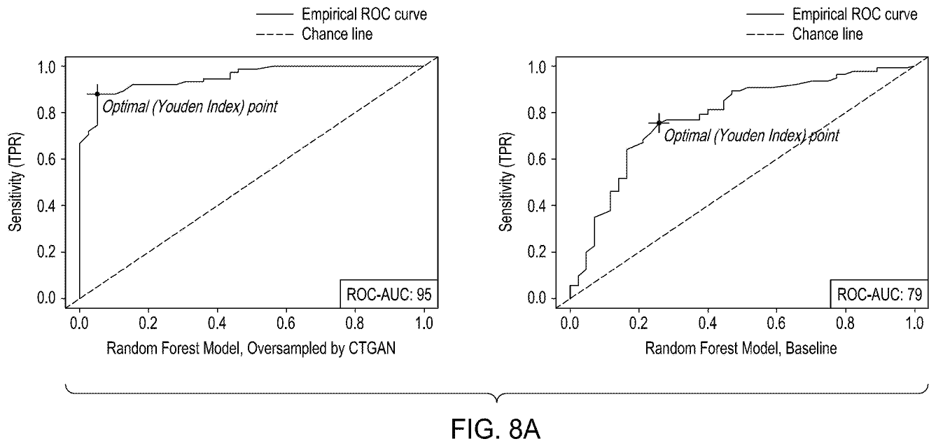

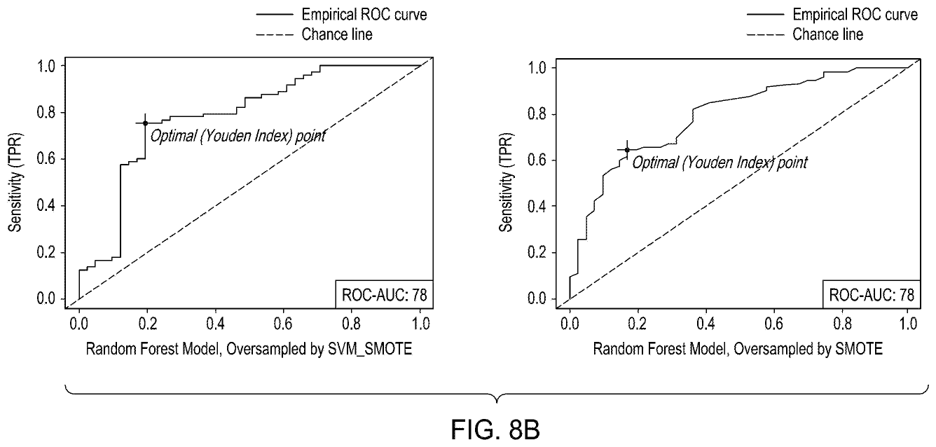

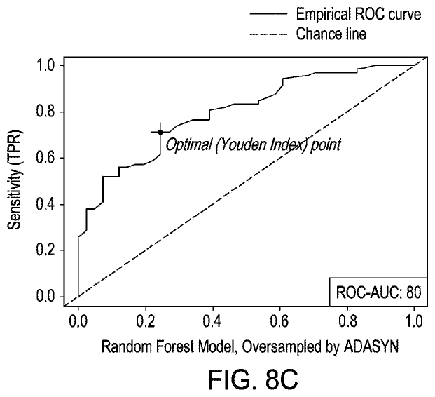

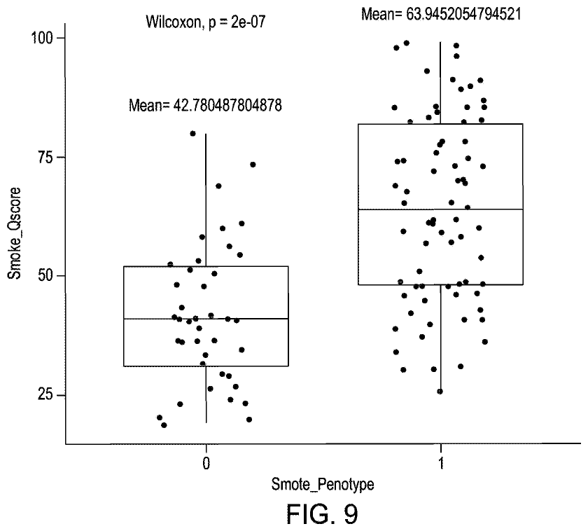

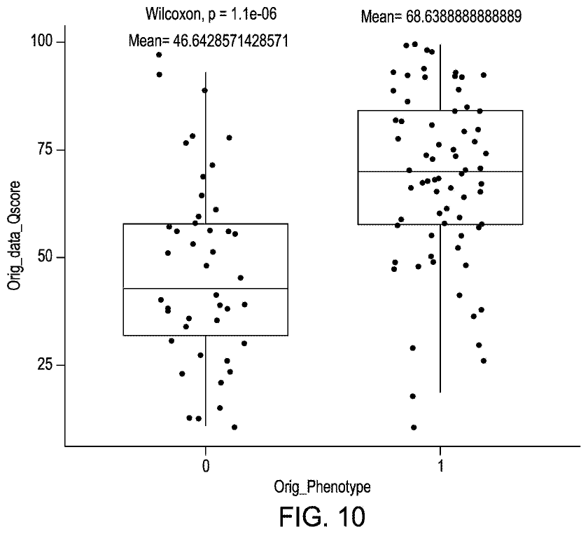

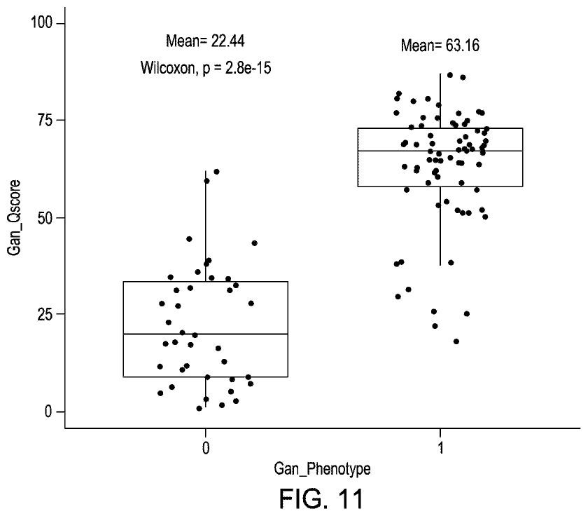

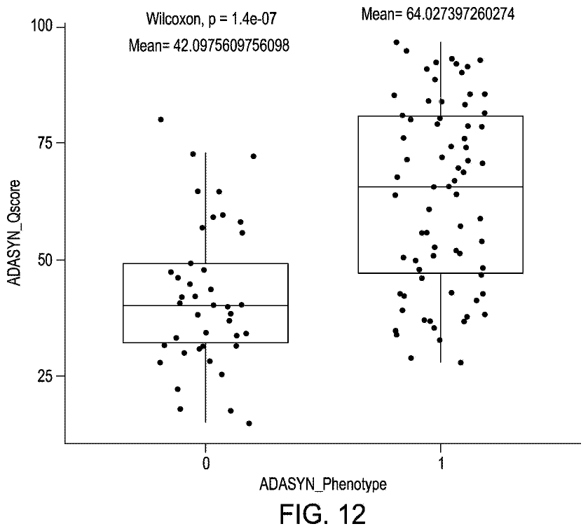

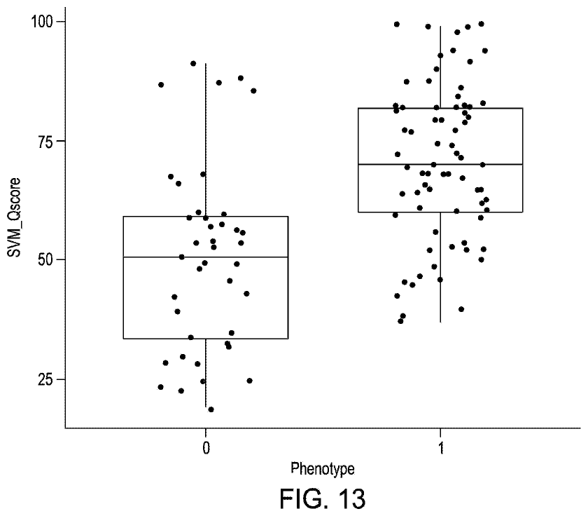

FIGS. 8A-8C, FIG. 9, FIG. 10, FIG. 11, illustrate non-parametric results of random forest-based kidney rejection scores using different synthetic data generation methods (0=Stable, 1=Acute Kidney Rejection). FIGS. 8A-8C collectively illustrate illustrates the performance of a random forest model oversampled by CTGAN and a baseline (FIG. 8A), a random forest model oversampled by SVM_SMOTE and SMOTE (FIG. 8B), and a random forest model oversampled by ADASYN (FIG. 8C), on kidney transplant rejection datasets with synthetic urine samples. FIG. 9 illustrates non-parametric results of random forest-based rejection scores using a SMOTE synthetic data generation method for providing a Q-Score. The axis of FIG. 9 represent the SMOTE generated Q-Score (Y-axis) over the SMOTE phenotype (X-axis). FIG. 10 illustrates non-parametric results of random forest-based rejection scores using original (i.e., biological) data generation method for providing a Q-Score. The axis of FIG. 10 represent the Q-Score of the original data (Y-axis) over the original phenotype. FIG. 11 illustrates non-parametric results of random forest-based rejection scores using a GAN synthetic data generation method for providing a Q-Score. The axis of FIG. 11 represent the GAN generated Q-Score (Y-axis) over the GAN phenotype (X-axis). FIG. 12 illustrates non-parametric results of random forest-based rejection scores using a ADASYN synthetic data generation method for providing a Q-Score. The axis of FIG. 12 represent the ADASYN generated Q-Score (Y-axis) over the ADASYN phenotype (X-axis). FIG. 13 illustrates non-parametric results of random forest-based rejection scores using a SVM synthetic data generation method for providing a Q-Score. The axis of FIG. 13 represent the SVM generated Q-Score (Y-axis) over the phenotype (X-axis).

While this invention is satisfied by embodiments in many different forms, as described in detail in connection with preferred embodiments of the invention, it is understood is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

What is claimed is:

1. A system configured to balance an imbalanced dataset obtained from a biological sample, comprising:
one or more computer subsystems; and one or more components executed by the one or more computer subsystems, wherein the one or more components comprise a generative adversarial network trained with:
a first training set comprising data corresponding to an amount of cell-free DNA (cfDNA) biomarker from a subject with an organ injury designated as a first training input;
a second training set comprising data corresponding to an amount of cell-free DNA (cfDNA) biomarker from a subject without the organ injury designated as a second training input;
wherein the first and the second datasets are imbalanced and the one or more computer subsystems are configured for generating a set of synthetic features for the first dataset and/or the second dataset by inputting a portion of the data from the first training input and the second training input into the generative adversarial network.

2. The system of claim 1, wherein the generative adversarial network is configured as a conditional generative adversarial network.

3. The system of claim 1, wherein the generative adversarial network is configured as a vanilla generative adversarial network.

4. The system of claim 1, wherein the generative adversarial network is configured as a table generative adversarial network.

5. The system of claim 1, wherein the generative adversarial network is configured as a tabular generative adversarial network.

6. The system of claim 1, wherein the generative adversarial network is further trained with an additional training set comprising data corresponding to an amount of a methylated cfDNA biomarker (m-cfDNA) from a subject with organ injury designated as an additional training input;
an additional training set comprising data corresponding to an amount of a methylated cfDNA biomarker (m-cfDNA) from a subject without organ injury designated as an additional training input.

7. The system of claim 1, wherein the generative adversarial network is further trained with an additional training set comprising data corresponding to an amount of an inflammatory biomarker from a subject with organ injury designated as an additional training input;
an additional training set comprising data corresponding to an amount of an inflammatory biomarker from a subject without organ injury designated as an additional training input.

8. The system of claim 7, wherein the inflammatory biomarker is a member of the chemokine (C—X—C motif) ligand family.

9. The system of claim 8, wherein the member of the chemokine (C—X—C motif) ligand family is C—X—C motif chemokine ligand 1 (CXCL1), C—X—C motif chemokine ligand 2 (CXCL2), C—X—C motif chemokine ligand 5 (CXCL5), C—X—C motif chemokine ligand 9 (CXCL9) (MIG), or C—X—C motif chemokine ligand 10 (CXCL10) (IP-10).

10. The system of claim 1, wherein the generative adversarial network is further trained with
an additional training set comprising data corresponding to an amount of an apoptosis biomarker from a subject with organ injury designated as an additional training input;
an additional training set comprising data corresponding to an amount of an apoptosis biomarker from a subject without organ injury designated as an additional training input.

11. The system of claim 10, wherein the apoptosis biomarker is clusterin.

12. The system of claim 1, wherein the generative adversarial network is further trained with
an additional training set comprising data corresponding to an amount of a protein from a subject with organ injury designated as an additional training input;
an additional training set comprising data corresponding to an amount of a protein from a subject without organ injury designated as an additional training input.

13. The system of claim 12, where the protein is albumin.

14. The system of claim 12, where the protein is total protein.

15. The system of claim 1, wherein the one or more computer subsystems are further configured for determining one or more characteristics of the synthetic features for the first dataset and/or the second dataset.

16. The system of any of claims 1-16, wherein the one or more computer subsystems are further configured to train a machine learning model using the simulated image.

17. The system of claim 16, wherein the machine learning model is trained on the first data input and on the second data input.

18. The system of claim 17, wherein the machine learning model is trained on the first data input and on the second data input, but not on the set of synthetic features.

19. The system of claim 16, wherein the machine learning model is CTGAN.

20. The system of claim 16, wherein the machine learning model is SMOTE.

21. The system of claim 16, wherein the machine learning model is SVM-SMOTE.

22. The system of claim 16, wherein the machine learning model is ADASYN.

23. The system of claim 1, wherein the biological sample is urine.

24. The system of claim 1, wherein the biological sample is blood.

25. The system of claim 1, wherein the organ is an allograft, and the injury is cause by rejection of the allograft by the subject.

26. The system of claim 1, wherein the organ is a kidney, a pancreas, a heart, a lung, or a liver.

27. The system of claim 26, wherein the organ is a kidney.

28. The system of claim 26, wherein the injury is chronic kidney injury (CKI) or acute kidney injury (AKI).

29. The system of claim 1, wherein the injury is caused by a viral infection suffered by the subject.

30. The system of claim 1, wherein the viral infection is caused by Sars-COV-2, CMV, or BKV.

31. The system of claim 1, wherein the injury is a cancer harming the organ.

32. The system of claim 1, wherein the subject is a human.

33. A system configured to analyze a dataset obtained from a biological sample, comprising:
one or more computer subsystems; and one or more components executed by the one or more computer subsystems, wherein the one or more components comprise a generative adversarial network trained with a training set corresponding to an amount of cfDNA from a subject; and wherein the one or more computer subsystems are configured for generating a synthetic dataset from the biological sample by inputting a subset of the training data into the generative adversarial network.

34. The system of claim 33, wherein the subset of the training data is annotated with a biological condition.

35. The system of claim 33, wherein at least one subset of the training data is annotated with a biological condition of acute rejection.

36. The system of claim 33, wherein at least one subset of the training data is annotated with a biological condition of chronic kidney injury (CKI) or acute kidney injury (AKI).

37. The system of claim 33, wherein at least one subset of the training data is annotated with a biological condition of COVID-19.

38. The system of claim 33, wherein at least one subset of the training data is annotated with a biological condition of healthy or stable.

39. The system of claim 33, wherein the cfDNA is from a urine sample.

40. The system of claim 33, wherein the cfDNA is from a blood or plasma sample.

41. The system of claim 33, wherein the generative adversarial network is further trained with an additional training set comprising data corresponding to an amount of a methylated cfDNA biomarker (m-cfDNA) from a subject.

42. The system of claim 33, wherein the generative adversarial network is further trained with an additional training set comprising data corresponding to an amount of an inflammatory biomarker from a subject.

43. The system of claim 42, wherein the inflammatory biomarker is a member of the chemokine (C—X—C motif) ligand family.

44. The system of claim 43, wherein the member of the chemokine (C—X—C motif) ligand family is C—X—C motif chemokine ligand 1 (CXCL1), C—X—C motif chemokine ligand 2 (CXCL2), C—X—C motif chemokine ligand 5 (CXCL5), C—X—C motif chemokine ligand 9 (CXCL9) (MIG), or C—X—C motif chemokine ligand 10 (CXCL10) (IP-10).

45. The system of claim 33, wherein the generative adversarial network is further trained with an additional training set comprising data corresponding to an amount of an apoptosis biomarker from a subject.

46. The system of claim 45, wherein the apoptosis biomarker is clusterin.

47. The system of claim 33, wherein the generative adversarial network is further trained with an additional training set comprising data corresponding to an amount of a protein.

48. The system of claim 47, where the protein is albumin.

49. The system of claim 47, where the protein is total protein.

50. The system of claim 33, wherein the subject is a human.

51. A non-transitory computer-readable medium, storing program instructions executable on one or more computer systems for performing a computer-implemented method for generating a simulated image of a specimen, wherein the computer-implemented method comprises:
one or more computer subsystems; and one or more components executed by the one or more computer subsystems, wherein the one or more components comprise a generative adversarial network trained with a training set corresponding to an amount of cfDNA from a subject; and wherein the one or more computer subsystems are configured for generating a synthetic dataset from the biological sample by inputting a sub-set of the training data into the generative adversarial network.

52. A non-transitory computer-readable medium, storing program instructions executable on one or more computer systems for performing a computer-implemented method for generating a simulated image of a specimen, wherein the computer-implemented method comprises:
one or more computer subsystems; and one or more components executed by the one or more computer subsystems, wherein the one or more components comprise a generative adversarial network trained with a training set comprising data corresponding to an amount of cell-free DNA (cfDNA) biomarker from a subject a first training set comprising data corresponding to an amount of cell-free DNA (cfDNA) biomarker from a subject with an organ injury designated as a first training input; a second training set comprising data corresponding to an amount of cell-free DNA (cfDNA) biomarker from a subject without the organ injury designated as a second training input;
wherein the first and the second datasets are imbalanced and the one or more computer subsystems are configured for generating a set of synthetic features for the first dataset and/or the second dataset by inputting a portion of the data from the first training input and the second training input into the generative adversarial network.
